(19) United States
(12) Patent Application Publication
(10) Pub. No.: US 2011/0144727 A1
(43) Pub. Date: Jun. 16, 2011

(54) PORTABLE PHOTOTHERAPY DEVICE AND METHOD FOR USING A PORTABLE THERAPY DEVICE

(76) Inventor: Mellen Thomas Benedict, Aptos, CA (US)

(21) Appl. No.: 12/638,735

(22) Filed: Dec. 15, 2009

(51) Int. Cl. A61N 5/06

(52) U.S. Cl. 607/91; 607/88

(57) ABSTRACT

The present invention is a portable and flexible phototherapy device that includes a light pad and a controller. The controller is preferably pre-programmed with several programs to allow the light therapy pad to be used in the comfort of the users home. The flexible phototherapy pad can be positioned over any area of a body in order to provide beneficial illumination of the body part.

### BACKGROUND OF THE INVENTION

This invention relates to a portable phototherapy device and method of using a portable phototherapy device. More specifically, the invention relates to a flexible phototherapy pad that can be positioned over any area of a body and that provides various beneficial illumination protocols.

Phototherapy involves generation of light by suitable light sources, such as light emitting diodes (LEDs) in the visible and infrared ranges to provide various benefits for a patient's body. The photons produced are absorbed by the body through the skin, the eyes, and acupuncture points. Connective tissues in the body conduct the light to deeper tissues and organs. By taking advantage of optical properties of biological tissues, suitable wavelengths of light can be delivered to, absorbed by, and used by the body to activate metabolic functions.

Typically, persons wishing to benefit from phototherapy must go to a spa or a professional health provider. This is because the phototherapy devices currently available are bulky and large. Additionally, until recently, a professional was needed to program and monitor a phototherapy session, because optimal parameters, such as wavelength range, relative distribution of the wavelengths within the range (spectrum), time interval for continuous exposure, time interval between two continuous exposures, time rate of energy delivered, accumulated energy density for exposures, and body component(s) irradiated, were not yet available. Now, many of the optimal parameters are better understood. For example, U.S. Pat. No. 6,524,329, issued to Mellen-Thomas Benedict (the inventor of the present invention), discloses a method of illuminating body components that provides beneficial treatment. Thus, it is now possible to provide a programmable portable phototherapy device that can be used in the comfort of a user's home and that can be adapted to various parts of the user's body. However, no such product currently exists.

In the field of portable phototherapy devices, United States Published Patent Application Number 2008/0255640, filed by Kipp et al., discloses a portable phototherapy device for treating skin conditions that is packable and can treat various parts of the body. However, Kipp discloses a device that is rigid, uses an ultraviolet blub, and has only one setting. Kipp does not disclose a flexible and programmable phototherapy device.

Thus, what is needed is a flexible, programable, and portable phototherapy device that can be used in the comfort of a users own home and that can be adapted to various parts of the users body.

### SUMMARY OF THE INVENTION

Various embodiments of the invention are directed towards overcoming the above deficiencies of the prior art by providing an easy-to-use, portable, programmable, and flexible phototherapy device.

One embodiment of the present invention is a portable phototherapy device comprising: a flexible light circuit layer; a controller; and a power supply. The flexible light circuit layer is connected to the controller. The flexible light circuit layer is comprised of a flexible circuit board, a circuit, and a plurality of lights. The controller is a programmable computing unit. The controller is programmed with one or more treatment modes. Preferably the plurality of lights are light emitting diodes. The portable phototherapy device may be positioned around a body component by flexibly curving said portable phototherapy device in an inward direction such that said portable phototherapy device partially encircles said body component. The treatment modes preferably comprise modulating said plurality of light emitting diodes with one or more frequency components. Preferably, the frequency components are faster than a human visual perception threshold. The plurality of light emitting diodes preferably include: blue diodes, yellow diodes, and red diodes. The frequency components may differ between said one or more treatment modes. The treatment modes may also include one or more trace patterns and selectively dimming said plurality of light emitting diodes. The diodes preferably dim based on a color of the light emitting diodes.

In another embodiment of the invention, the portable phototherapy device also includes a front pad layer, which is attached to the front of the flexible light circuit layer. The front pad layer is perforated with a plurality of holes. These holes allow light from the flexible light circuit layer to pass through the front pad layer and illuminate the body component. The portable phototherapy device may also include a back support layer, which is attached to said back of said flexible light circuit layer. In this embodiment the lights are preferably light emitting diodes. The treatment modes comprise modulating the plurality of light emitting diodes with one or more frequency components and the frequency components are faster than a human visual perception threshold. The light emitting diodes are preferably blue diodes, yellow diodes, and red diodes. Preferably the frequency components differ between said one or more treatment modes. The treatment modes preferably include one or more trace patterns and selectively dimming the light emitting diodes. The diodes are programmed to dim based on a color of said light emitting diodes. The portable phototherapy device preferably includes a shield layer and a body strap. The shield layer is attached to a back of said back support layer. The portable phototherapy device is preferably positioned around a body component by flexibly curving the portable phototherapy device in an inward direction such that the portable phototherapy device partially encircles said body component. The body strap holds the positioned portable phototherapy device around said body component. The back support layer preferably includes a plurality of ridges. These ridges allow a portable phototherapy device to be flexibly curved in an inward direction.

Another embodiment of the invention is a method of illuminating a body component, the method comprising: providing a portable phototherapy device; wherein said portable phototherapy device is comprised of a flexible light circuit layer, a controller, a front pad layer, a back support layer, and a power supply; wherein said flexible light circuit layer is comprised of a flexible circuit board, a circuit, and a plurality of light emitting diodes; wherein said plurality of light emitting diodes are comprised of blue diodes, yellow diodes, and red diodes; wherein said flexible light circuit layer is connected to said controller; wherein said flexible light circuit layer has a front and a back; wherein said controller comprises a computing unit; wherein said controller is programmable; wherein said controller is programmed with one or more treatment modes; wherein said back support layer is attached to said back of said flexible light circuit layer; wherein said front pad layer is attached to said front of said flexible light circuit layer; wherein said front pad layer is perforated with a plurality of holes; and positioning said portable phototherapy device around a body component by flexibly curving said portable phototherapy device in an inward direction such that said portable phototherapy device partially encircles said body component; wherein said plurality of holes of said front pad layer allow a light from said plurality of light emitting diodes to illuminate said body component; selecting at least one of said one or more treatment modes using said controller; activating said selected one or more treatment modes using said controller; and illuminating said body component.

The method of illuminating a body component preferably includes: modulating said light emitting diodes with one or more frequency components during said selected one or more treatment modes; wherein said one or more frequency components are faster than a human visual perception threshold; wherein said one or more frequency components differ between said one or more treatment modes; activating one or more trace patterns during said selected one or more treatment modes; and dimming one or more of said plurality light emitting diodes based on a color of said light emitting diodes during said one or more treatment modes.

The invention is a portable and flexible phototherapy device that is pre-programmed with several treatment modes so that a user can safely and easily use the device in the comfort of their own home. The invention is comprised of a pad, a controller, and a power supply. Preferably the pad is composed of at least three layers: a back layer, a front layer, and a middle layer. The back layer and the front layer preferably have identical outlines and are made out of an elastic material that allows the identical outlines to remain even when the invention is formed into an arc. The middle layer has an outline that is preferably smaller than the outline of the back and front layer. The middle layer is preferably printed circuit board (PCB) with a printed circuit and light array on the board. The middle layer is placed between the back and front layer, which are heat sealed together. The front layer has an array of holes that align with the light array of the middle layer so that the light from the light array shines through the front layer. The front layer is padded so that the user is comfortable when engaging with the phototherapy device. The back layer is supportive and preferably includes ridges which allow the phototherapy device to easily bend inward, but resist bending backward.

The power supply can be an AC adaptor or a battery.

The controller is preferably an easy to use device that lets the user operate the machine in several different therapeutic modes. The modes are preprogrammed and typically include a modulated frequency component, which flashes faster than the human visual perception threshold, a selective dimming protocol, and a trace pattern. Depending upon which mode the user selects determines what the light emitting diodes, the preferred lights of the light array, do. The preprogrammed modes can be altered by the user so that the treatment may be customized to a specific user. Additionally, the controller can allow the user to program in their own treatment mode from scratch.

An object of the present invention is to provide a phototherapy device that overcomes the limitations of the prior art.

Another object of the present invention is to provide a portable phototherapy device that is programmable and comes with several preprogrammed treatment modes.

Another object of the present invention is to provide a phototherapy device that is portable and easy to use without the need for a professional to set the treatment modes before each use.

Another object of the present invention is to provide a self-contained, compact, portable, and inexpensive phototherapy device that can engage and effectively illuminate all parts of a body.

Other features and advantages are inherent in the phototherapy device claimed and disclosed will become apparent to those skilled in the art from the following detailed description and its accompanying drawings.

### BRIEF DESCRIPTION OF THE DRAWINGS

FIG. 1 is a perspective back view of the pad of one embodiment of the invention.

FIG. 2 is a perspective front view of the pad of one embodiment of the invention.

FIG. 3 is a perspective view of the pad of one embodiment of the invention and shows the pad flexed inward.

FIG. 4 is a cross section view of the pad of one embodiment of the invention and shows the three layers of the pad and how the edges of the layers of the pad are sealed together.

FIG. 5 is an exploded view of the pad of one embodiment of the invention and shows how the three layers of the pad are arrayed together.

FIG. 6 is a perspective view of one embodiment of the invention and shows the shield.

FIG. 7 is a perspective view of one embodiment of the invention and shows the shield, the power supply, and the body strap.

FIG. 8 is an front view of the light array of one embodiment of the invention.

FIG. 9 is a top view of the controller of one embodiment of the invention.

### DETAILED DESCRIPTION OF THE DRAWINGS

In the following detailed description of the various embodiments of the invention, numerous specific details are set forth in order to provide a thorough understanding of various embodiments of the invention. However, one or more embodiments of the invention may be practiced without these specific details. In other instances, well-known methods, procedures, and/or components have not been described in detail so as not to unnecessarily obscure aspects of embodiments of the invention.

In the following detailed description of the various embodiments of the invention, reference is made to the accompanying drawings that form a part hereof, and in which is shown by way of illustration a specific embodiment in which the invention may be practiced. It is to be understood that other embodiments may be utilized and structural changes may be made without departing from the scope of the present invention. Accordingly, the drawings and detailed description are to be regarded as illustrative in nature and not restrictive. In addition, the reference or non-reference to a particular embodiment of the invention shall not be interpreted to limit the scope of the invention.

In the following description, certain terminology is used to describe certain features of one or more embodiments of the invention. For instance “circuit board” or “printed circuit board” refers to any single-sided, double-sided, or multi-layered board that includes at least one conductive layer and at least one insulating or substrate layer. The circuit board preferably includes at least one or more lights.

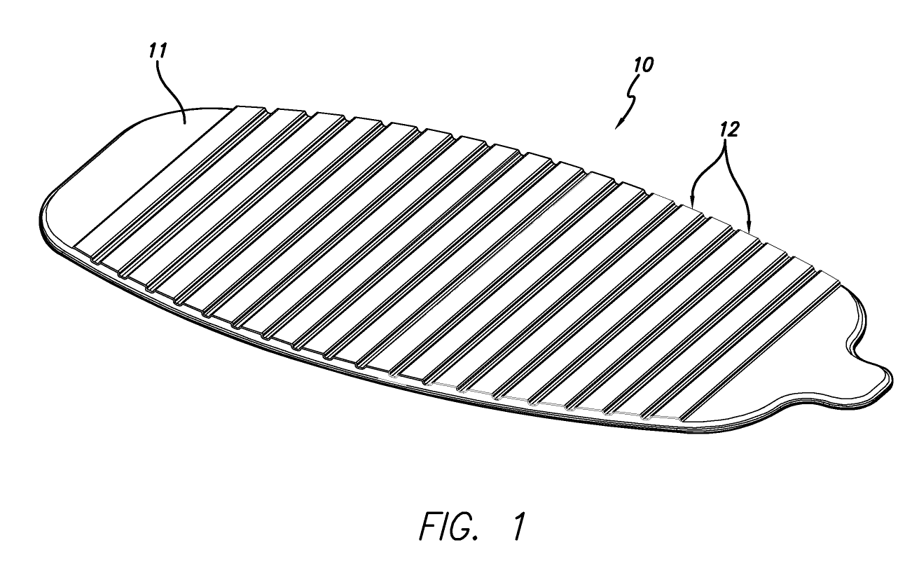

FIG. 1 is a perspective back view of the pad of one embodiment of the invention. As shown in FIG. 1, the invention preferably includes a layered light pad 10. FIG. 1 shows that the back of the light pad preferably is a back support layer 11 with ridges 12. Although the back support layer 11 is preferably made of a flexible yet rigid foam, the back support layer may be made from any natural or man-made material.

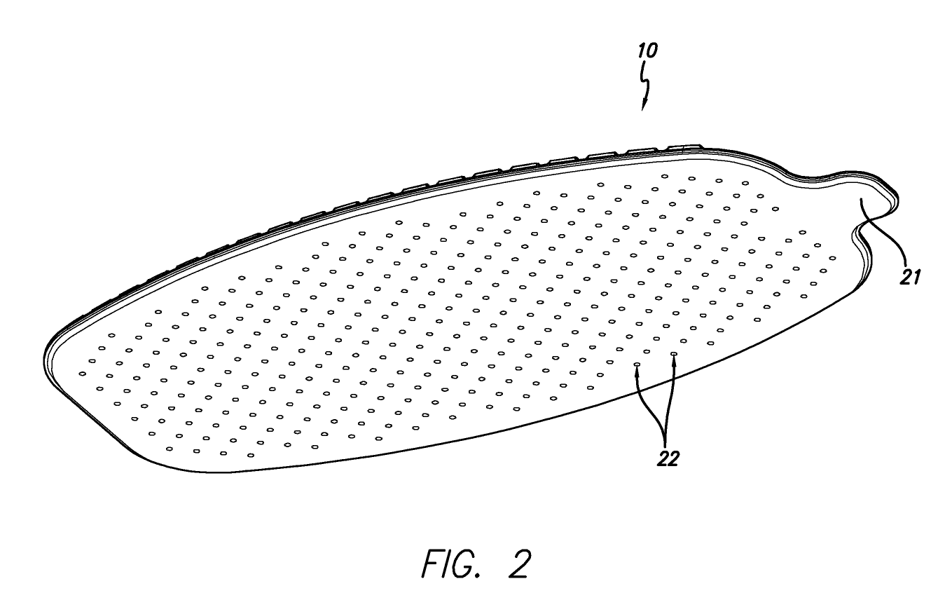

FIG. 2 is a perspective front view of the pad of one embodiment of the invention. As shown in FIG. 2, the invention preferably includes a layered light pad 10 with a soft and perforated front layer 21. FIG. 2 shows that the front layer 21 preferably has dozens of holes 22 or perforations. These holes 22 allow light from a middle layer to shine through to the user of the light pad 10. Although the front layer 21 is preferably made of a soft and flexible foam, the front layer 21 may be made from any natural or man-made material.

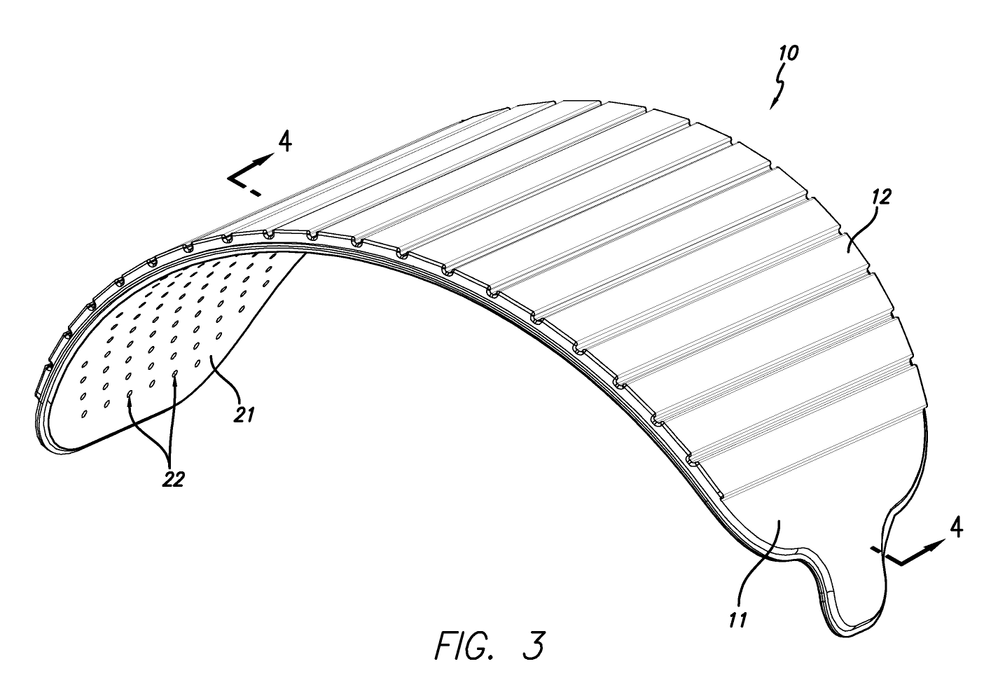

FIG. 3 is a perspective front view of the pad of one embodiment of the invention and shows the pad flexed inward. FIG. 3 shows that the light pad 10 as being flexible, especially in an inward direction. The light pad 10 is shown with back support layer 11, ridges 12, front layer 21, and holes 22. FIG. 3 shows how back support layer 11 and front layer 21 are preferably cut to have identical footprints or outlines. This allows the back support layer 11 and front layer 21 to be sealed together. Preferably back support layer 11 and front layer 21 are made from a flexible and stretchable (or elastic) material that allows them to bend inward without bunching and without putting unnecessary stress on the sealed edges. In this manner, the back support layer and front layer maintain essentially identical footprints regardless of how the pad is twisted or bent.

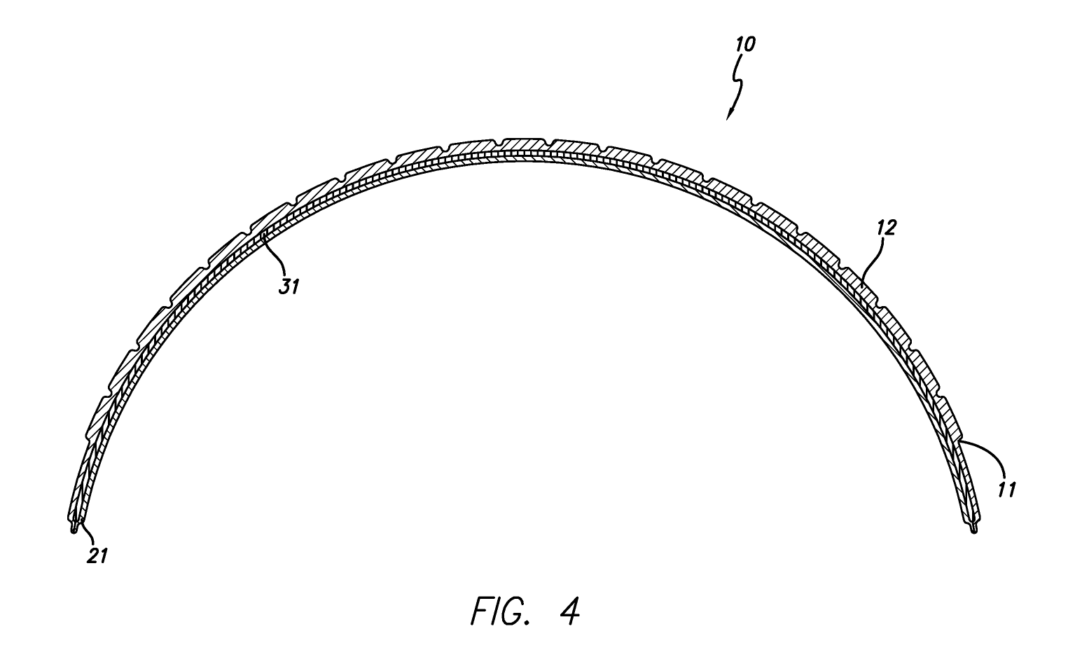

FIG. 4 is a cross section view of the pad of one embodiment of the invention and shows the three layers of the pad and how the edges of the layers of the pad are sealed together. As shown in FIG. 4, light pad 10 preferably includes three (3) layers: back support layer 11, which preferably has ridges 12; front layer 21, which is preferably perforated (not shown in FIG. 4); and middle layer 31, which is preferably a light circuit board.

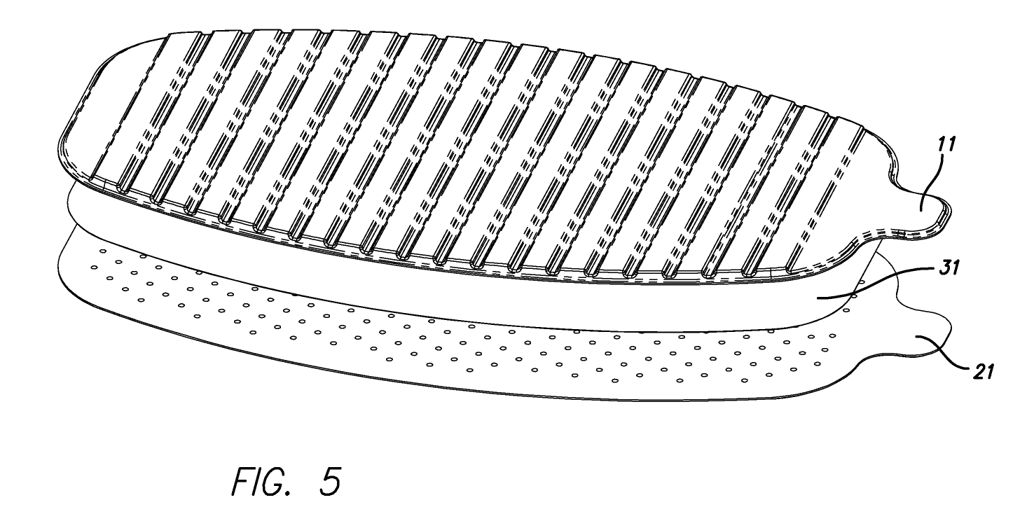

FIG. 5 is an exploded view of the pad of one embodiment of the invention and shows how the three layers of the pad are arrayed together. As shown in FIG. 5, the pad preferably includes back support layer 11, front layer 21, and middle layer 31. Middle layer 31 preferably has a slightly smaller footprint. This allows the back support layer 11 and front layer 21 to completely encase middle layer 31.

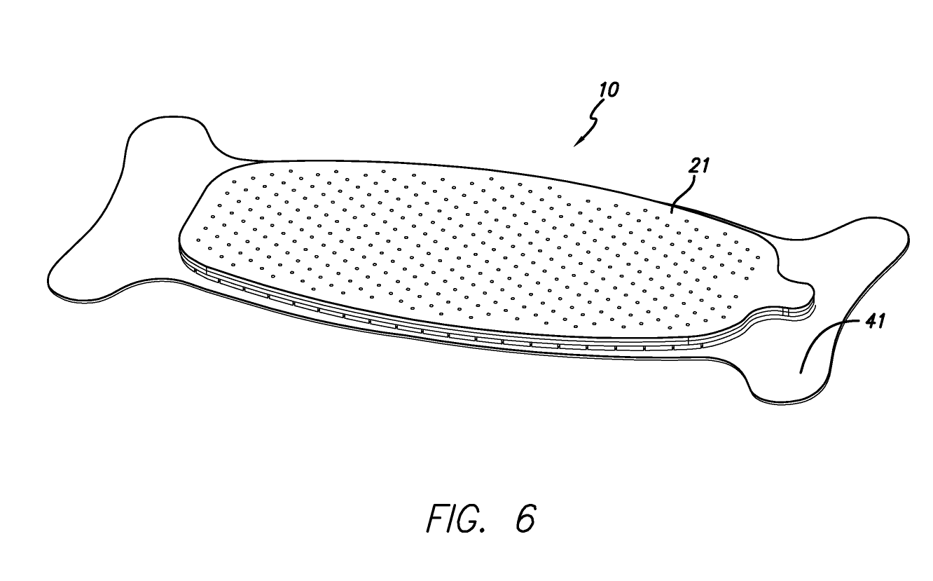

FIG. 6 is a perspective view of one embodiment of the invention and shows the shield. FIG. 6 shows how light pad 10 preferably has a shield layer 41 connected to the back side. Shield layer 41 is preferably a poly carbonate layer. The shield layer 41 provides additional rigidity to light pad 10. Although shield layer 41 is preferably made out of a clear plastic material, it can be made from any natural or man-made material without deviating from the scope of the invention.

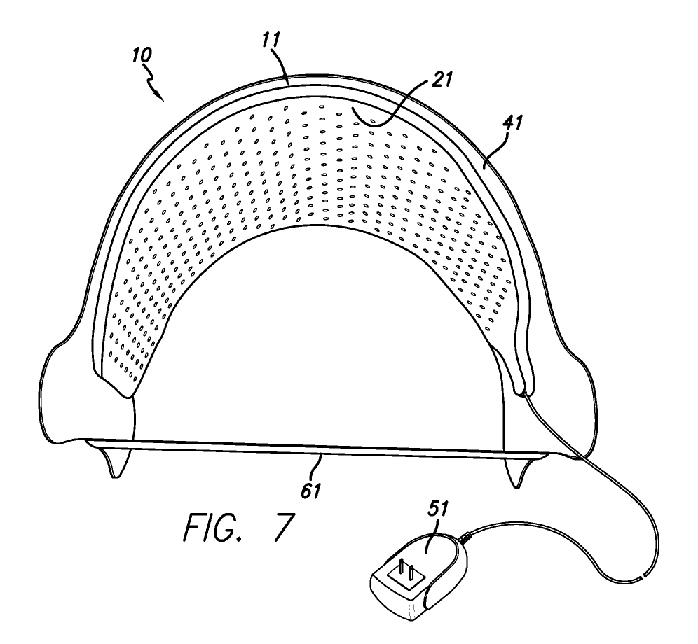

FIG. 7 is a perspective view of one embodiment of the invention and shows the shield, the power supply, and the body strap. As shown in FIG. 7, the light pad 10 preferably includes front layer 21, back support layer 11, shield layer 41, power supply 51, and body strap 61. FIG. 7 shows that the pad is connected to power supply 51, which may be connected to an A/C source, such as a wall outlet. Alternatively, the power supply may be a D/C battery. Additionally, power supply 51 is preferably connected to a controller (not shown in FIG. 7). FIG. 7 shows body strap 61 being used to arc or bend light pad 10 such that light pad 10 may partially encircle and remain fixed around a body part during a light treatment session. Although FIG. 7 shows that the body strap 61 is preferably a flexible strap, the body strap 61 may be any device that bends light pad 10 around a body part and holds it there.

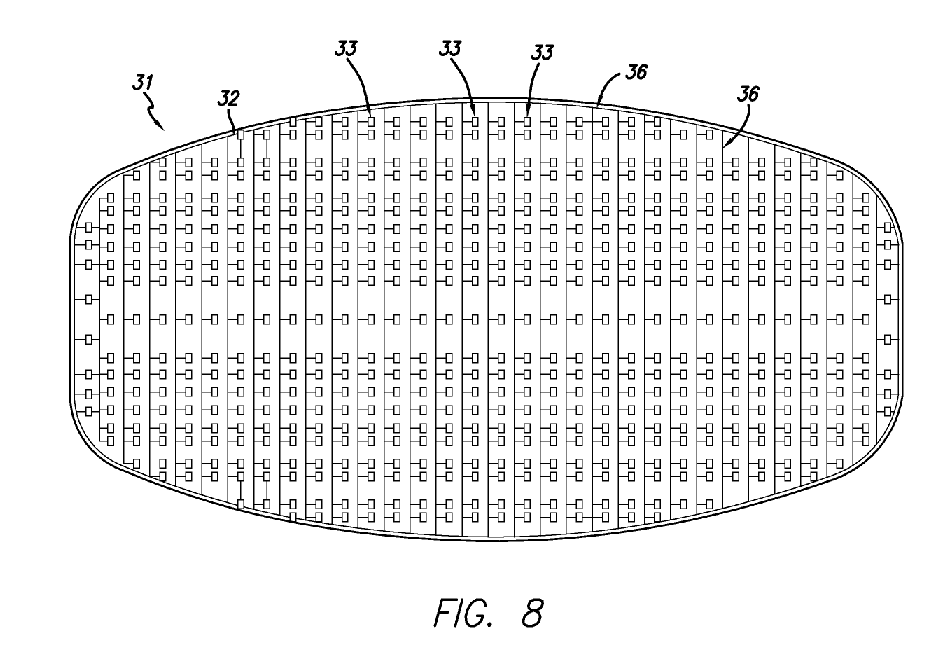

FIG. 8 is an front view of the light array of one embodiment of the invention. FIG. 8 shows that middle layer 31 is preferably a flexible light array circuit board that includes circuit board 32, lights 33, and printed circuit 36. Preferably the printed circuit connects all of the lights together and to a controller (not shown in FIG. 8). Preferably circuit board 32 is a standard flexible substrate board. FIG. 8 shows that the lights 33 are preferably light emitting diodes and are available in many different colors, including blue, yellow, and red (the primary colors). The lights 33 on middle layer 31 are preferably arrayed with the holes of the front layer (not shown in FIG. 8). This allows light from the lights 33 to shine through the front layer and onto a body part of the user. Although FIG. 8 shows that there are preferably dozens and dozens of lights, the invention may have any number of lights without deviating from the scope of the invention. Additionally, although, as preferred, FIG. 8 shows that the lights are arrayed in columns from the left to the right, the lights may be in any array without deviating from the scope of the invention.

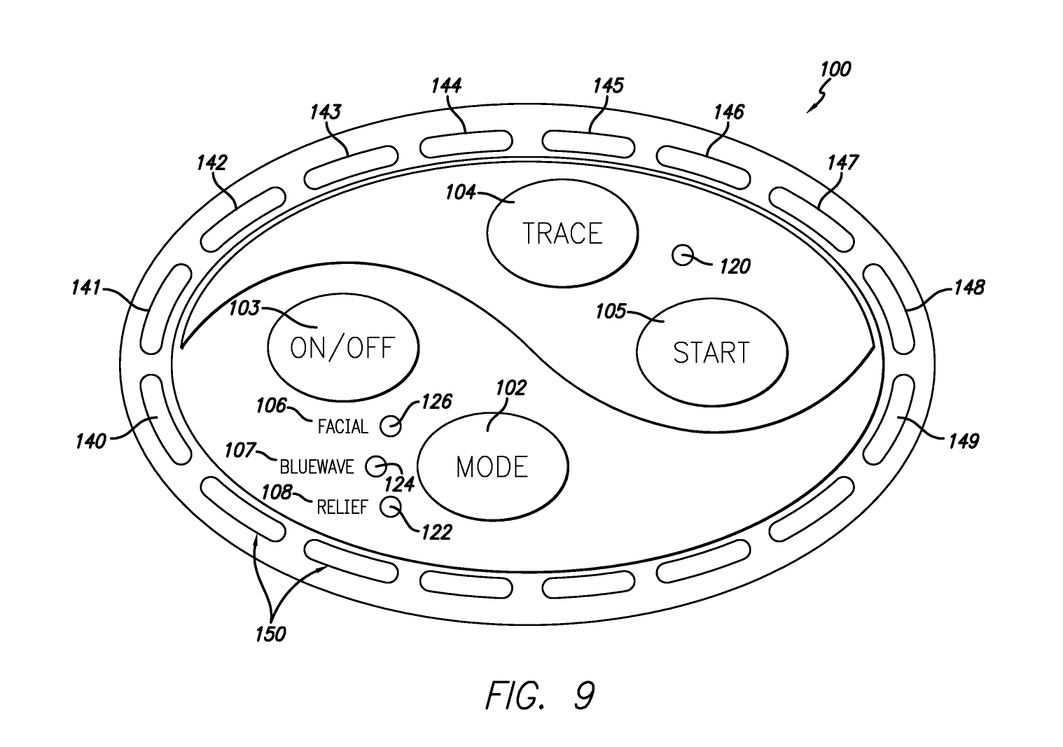

FIG. 9 is a top view of the controller of one embodiment of the invention. FIG. 9 shows that controller 100 preferably includes mode button 102, on/off button 103, trace button 104, start button 105, facial mode display 106, bluewave mode display 107, relief mode display 108, tracking lights 140, 141, 142, 143, 144, 145, 146, 147, 148, 149, auxiliary lights 150, and indicator lights 120, 122, 124, and 126. Controller 100 preferably includes a programmable computing unit that may include several pre-programmed modes. As shown in FIG. 9 the controller is accessed by pushing the mode button 102, on/off button 103, trace button 104, and start button 105. Although buttons are shown in FIG. 9, it should be understood that any selection means could be used, such as switches, without deviating from the scope of the invention.

Preferably the controller 100 includes two standard circular connectors, which may be rectangular or any other shape without deviating from the scope of the invention. One is to connect to the power supply and the other is to connect to the light pad. The controller 100 is used to command or control the light pad. Alternatively, the controller may control the light pad wirelessly and may be powered by batteries.

The Pre-Programmed Treatment Modes.

Preferably the controller 100 is preprogrammed with at least three treatment modes. FIG. 9 shows that these modes are preferably referred to with fanciful names, such as, Facial™ Bluewave™, and Relief™. Preferably the programmed modes are different from each other so that the user can access different phototherapy treatments depending on their needs. Although the treatment modes may be programmed to run for any duration of time, the preferred treatment mode time is thirty (30) minutes. The preferred light source for the phototherapy device of the present invention is a light emitting diode (LED). Although FIG. 9 shows that the present invention preferably includes three treatment modes, and these treatment modes have suggestive names, it should be understood that the present invention may have any number of preprogrammed treatment modes, that the treatment modes can be altered to accommodate the specific users preferences, and that the treatment modes can be named anything.

The Frequency Component. Preferably during the treatment modes, some or all of the LEDs are modulated with a frequency component, which is above, or faster than, the human visual perception threshold. In other words, although the LEDs are blinking, they are blinking at a rate that is too fast to be visible. The frequency component preferably varies with each mode and the frequency preferably changes at different times during the treatment. Additionally, there are preferably periods of no frequency during the treatment. During these periods of no frequency the LEDs are on constantly. The duty cycle of the frequency component has a preferred minimum of 90%, which means that the LEDs are on for 90% of the time and off for the other 10% of the cycle. This duty cycle may be varied to effect the fading up and/or fading down of groups of LEDs or all of the LEDs.

The Trace Pattern. Preferably the treatment modes also include a visible trace rate, which appears as a moving pattern that moves horizontally across the pad and preferably varies in rate from 40 Hertz (Hz) to 5.0 Hz. This is preferably done by programming the switching off of successive columns of LEDs in a sequential manner. This allows the majority of the LEDs on the pad to remain on while creating a trace pattern effect. Preferably the trace pattern is usually on, but the user can switch it off by pushing the trace 120 button. The visible trace pattern contributes to brain entertainment and relaxation. An important difference between the trace rate and the frequency component is that all of the LEDs preferably have the same frequency component at the same time, regardless of which treatment mode is selected, but, preferably, only specific LEDs may have the trace component, based on the selected treatment mode. The trace may trace a group of LEDs, such as red alone, red and blue, or yellow and red, or the entire LED array.

The preferred trace pattern rates during a preferred thirty (30) minute treatment range from 40 Hz to 5 Hz and may be intentionally varied within this range over the treatment period. For example the first 15 minutes may be at 30 Hz and the last 15 minutes may be at 13 Hz. The trace pattern rates may be the same for all modes (Facial™, Bluewave™, and Relief™) or may be different for each mode. It should be understood that any visible trace pattern rate could be used and the rate may go up to down, down to up, or both during the treatment session without deviating from the scope of the invention.

LED Intensity. Preferably the treatment modes also differ in the intensity of which the various LED colors are presented. For example, in one treatment mode the blue LEDs may be dimmed. In another the red and yellow LEDs may be dimmed or faded up and down in a sinusoidal manner over several seconds. The selection of which LEDs to dim or fade up and down depends on what the treatment is trying to accomplish.

Preferably the different treatment modes are programmed with varying treatment frequency ranges. For example, one of the preferred treatment modes, such as Facial™, may be programmed to include frequencies in the range of 1,000 Hz to 200 Hz. The frequencies may vary throughout the Facial™ treatment period within the programmed range. In addition, periods of no frequency, or constant on, are preferably interspersed between frequency changes. Preferably, the Facial™ treatment mode is programmed so that the red and yellow LEDs are used to accomplish the trace pattern and the blue LEDs are dimmed in intensity for the duration of the treatment.

Preferably the Relief™ treatment mode is programmed to include frequencies in the range of 300 Hz to 60 Hz and frequencies vary throughout the treatment period within this range. In addition, periods of no frequency, constant on, are preferably interspersed between frequency changes. Preferably, the relief treatment mode is programmed so that the blue LEDs are used to accomplish the trace pattern and the red and yellow LEDs are faded up and down in intensity for the duration of the treatment.

Preferably the Bluewave™ treatment mode is programmed to include to include frequencies in the range of 1,000 Hz to 400 Hz and frequencies vary throughout the treatment period within this range. In addition periods of no frequency (constant on) are interspersed between frequency changes. Preferably, the Bluewave™ treatment mode is programmed so that the red and yellow LEDs are used to accomplish the trace pattern and the same red and yellow LEDs are dimmed in intensity for the duration of the treatment.

Fade In and Fade Out. Finally, it is preferred that during the first 20 seconds of any treatment mode the LEDs start out very dim (1% intensity) and fade up over the those first 20 seconds to full intensity. It is also preferred that during the last 20 seconds the LEDs fade back down to a dim 1% intensity and then switch off. These fade ups and fade downs may be accomplished by varying the frequency duty cycle from 1% to 90% and from 90% to 1%. However, the user may stop the treatment at any time by pushing the start 105 button or the on/off 103 button.

The tracking lights 140, 141, 142, 143, 144, 145, 146, 147, 148, 149 preferably illuminate to show the progress of a selected treatment. For example the tracking of the tracking lights may represent three (3) minutes of treatment, so when the first nine (9) of the bars are illuminated, there is three minutes or less remaining in the thirty (30) minute treatment. Additionally, the auxiliary lights 150, which are not used to represent three (3) minutes of treatment can be used to show progress through the next three minutes.

Auxiliary Functions.

Preferably, the present invention is programmable with several auxiliary functions, including but not limited to: combining two or more of the treatment modes; altering the time of the treatment mode; altering the frequencies; altering the trace pattern rates; altering the dimming components; and programming the invention to run multiple sessions over an extended period of time (burn-in mode). Preferably the additional programming abilities and auxiliary functions are accessed by pushing the mode 102 button in a specific pattern. For example, to combine the preprogrammed modes and create a “combo” treatment mode the user uses the following steps:

1) Start with the system off.
2) Press and hold the mode 102 button for five seconds and then release.
3) Upon release, indicator lights 122, 124, and 126 will be illuminated to indicate that all three modes will be part of the treatment.
4) Press start the start 105 button.
5) Facial will run for ten (10) minutes, then relief for ten (10) minutes and then blue wave for ten (10) minutes.
6) The appropriate indicator lights 122, 124, and 126 will illuminate depending on which treatment is being conducted.
If the mode 102 button is pushed again before the start button, the user can program a different combination.

Although the present invention is preferably programmed with several treatment modes and these treatment modes preferably include a frequency component, a dimming component, and a trace pattern, the treatment modes may be programmed without any or all of the listed components without deviating from the scope of the invention.

The foregoing description of the preferred embodiment of the invention has been presented for the purposes of illustration and description. While multiple embodiments are disclosed, still other embodiments of the present invention will become apparent to those skilled in the art from the above detailed description, which shows and describes illustrative embodiments of the invention. As will be realized, the invention is capable of modifications in various obvious aspects, all without departing from the spirit and scope of the present invention. Accordingly, the detailed description is to be regarded as illustrative in nature and not restrictive. Also, although not explicitly recited, one or more embodiments of the invention may be practiced in combination or conjunction with one another. Furthermore, the reference or non-reference to a particular embodiment of the invention shall not be interpreted to limit the scope of the invention. It is intended that the scope of the invention not be limited by this detailed description, but by the claims and the equivalents to the claims that are appended hereto.

We claim:

1. A portable phototherapy device comprising:
a flexible light circuit layer;
a controller; and
a power supply;
wherein said flexible light circuit layer is connected to said controller;
wherein said flexible light circuit layer is comprised of a flexible circuit board, a circuit, and a plurality of lights;
wherein said controller comprises a computing unit;
wherein said controller is programmable; and
wherein said controller is programmed with one or more treatment modes.

2. The portable phototherapy device of claim 1, wherein said plurality of lights are light emitting diodes.

3. The portable phototherapy device of claim 2, wherein said portable phototherapy device is positioned around a body component by flexibly curving said portable phototherapy device in an inward direction such that said portable phototherapy device partially encircles said body component.

4. The portable phototherapy device of claim 3, wherein said one or more treatment modes comprise modulating said plurality of light emitting diodes with one or more frequency components.

5. The portable phototherapy device of claim 4, wherein said one or more frequency components are faster than a human visual perception threshold.

6. The portable phototherapy device of claim 5, wherein said plurality of light emitting diodes include blue diodes, yellow diodes, and red diodes.

7. The portable phototherapy device of claim 4, wherein said one or more frequency components differ between said one or more treatment modes.

8. The portable phototherapy device of claim 4, wherein said one or more treatment modes include one or more trace patterns.

9. The portable phototherapy device of claim 4, wherein said one or more treatment modes include selectively dimming said plurality of light emitting diodes.

10. The portable phototherapy device of claim 9, wherein said plurality of diodes are programmed to selectively dim based on a color of said light emitting diodes.

11. The portable phototherapy device of claim 1, further comprising:
a front pad layer;
wherein said flexible light circuit layer has a front and a back;
wherein said front pad layer is attached to said front of said flexible light circuit layer;
wherein said front pad layer is perforated with a plurality of holes; and
wherein said plurality of holes of said front pad layer allow a light from said plurality of lights of said flexible light circuit layer to pass through said front pad layer and illuminate said body component.

12. The portable phototherapy device of claim 11, further comprising:
a back support layer;
wherein said back support layer is attached to said back of said flexible light circuit layer.

13. The portable phototherapy device of claim 12, wherein said plurality of lights are light emitting diodes;
wherein said one or more treatment modes comprise modulating said plurality of light emitting diodes with one or more frequency components;
wherein said one or more frequency components are faster than a human visual perception threshold; and
wherein said plurality of light emitting diodes include blue diodes, yellow diodes, and red diodes.

14. The portable phototherapy device of claim 13, wherein said one or more frequency components differ between said one or more treatment modes.

15. The portable phototherapy device of claim 14, wherein said one or more treatment modes include one or more trace patterns; and
wherein said one or more treatment modes include selectively dimming said plurality of light emitting diodes.

16. The portable phototherapy device of claim 15, wherein said plurality of diodes are programmed to selectively dim based on a color of said light emitting diodes.

17. The portable phototherapy device of claim 16, further comprising:
a shield layer; and
a body strap;
wherein said shield layer is attached to a back of said back support layer;
wherein said portable phototherapy device is positioned around a body component by flexibly curving said portable phototherapy device in an inward direction such that said portable phototherapy device partially encircles said body component;
wherein said body strap is attached to said shield layer and holds said positioned portable phototherapy device around said body component.

18. A portable phototherapy device comprising:
a flexible light circuit layer;
a controller;
a front pad layer;
a back support layer;
a power supply;
a shield layer; and
a body strap;
wherein said flexible light circuit layer is comprised of a flexible circuit board, a circuit, and a plurality of lights;
wherein said plurality of lights are light emitting diodes;
wherein said plurality of light emitting diodes include blue diodes, yellow diodes, and red diodes;
wherein said flexible light circuit layer is connected to said controller;
wherein said flexible light circuit layer has a front and a back;
wherein said controller comprises a computing unit;
wherein said controller is programmable;
wherein said controller is programmed with one or more treatment modes;
wherein said one or more treatment modes comprise modulating said light emitting diodes with one or more frequency components;
wherein said one or more frequency components are faster than a human visual perception threshold;
wherein said one or more frequency components differ between said one or more treatment modes;
wherein said one or more treatment modes include one or more trace patterns;
wherein said one or more treatment modes include selectively dimming said plurality of light emitting diodes;
wherein said plurality of diodes are programmed to selectively dim based on a color of said light emitting diodes;
wherein said back support layer is attached to said back of said flexible light circuit layer;
wherein said back support layer comprises a plurality of ridges;
wherein said front pad layer is attached to said front of said flexible light circuit layer;
wherein said front pad layer is perforated with a plurality of holes; and
wherein said plurality of ridges allow a portable phototherapy device to be flexibly curved in an inward direction;
wherein said portable phototherapy device is positioned around a body component by flexibly curving in said inward direction such that said portable phototherapy device partially encircles said body component;
wherein said plurality of holes of said front pad layer allow a light from said plurality of lights of said flexible light circuit layer to illuminate said body component;
wherein said shield layer is attached to a back of said back support layer; and
wherein said body strap is attached to said shield layer and holds said positioned portable phototherapy device around said body component.

19. A method of illuminating a body component, the method comprising:
providing a portable phototherapy device;
wherein said portable phototherapy device is comprised of a flexible light circuit layer, a controller, a front pad layer, a back support layer, and a power supply;
wherein said flexible light circuit layer is comprised of a flexible circuit board, a circuit, and a plurality of light emitting diodes;
wherein said plurality of light emitting diodes are comprised of blue diodes, yellow diodes, and red diodes;
wherein said flexible light circuit layer is connected to said controller;
wherein said flexible light circuit layer has a front and a back;
wherein said controller comprises a computing unit;
wherein said controller is programmable;
wherein said controller is programmed with one or more treatment modes;
wherein said back support layer is attached to said back of said flexible light circuit layer;
wherein said front pad layer is attached to said front of said flexible light circuit layer;
wherein said front pad layer is perforated with a plurality of holes; and
positioning said portable phototherapy device around a body component by flexibly curving said portable phototherapy device in an inward direction such that said portable phototherapy device partially encircles said body component;
wherein said plurality of holes of said front pad layer allow a light from said plurality of light emitting diodes to illuminate said body component;
selecting at least one of said one or more treatment modes using said controller;
activating said selected one or more treatment modes using said controller; and
illuminating said body component.

20. The method of illuminating a body component of claim 19, the method further comprising:
modulating said light emitting diodes with one or more frequency components during said selected one or more treatment modes;
wherein said one or more frequency components are faster than a human visual perception threshold;
wherein said one or more frequency components differ between said one or more treatment modes;
activating one or more trace patterns during said selected one or more treatment modes; and
dimming one or more of said plurality light emitting diodes based on a color of said light emitting diodes during said one or more treatment modes.
